(19) United States
(12) Patent Application Publication
(10) Pub. No.: US 2026/0204070 A1
(43) Pub. Date: Jul. 16, 2026

(54) METHODS FOR VIDEO-BASED IMAGE SEGMENTATION AND APPLICATIONS THEREOF

(71) Applicant: Alpha Intelligence Manifolds, Inc., New Taipei City (TW)

(72) Inventor: CHIA-HUNG LIN, New Taipei City (TW)

(21) Appl. No.: 19/448,064

(22) Filed: Jan. 13, 2026

Related U.S. Application Data
(60) Provisional application No. 63/744,827, filed on Jan. 13, 2025.

(51) Int. Cl. G06V 20/40; G06V 10/77; G06V 10/774; G06V 10/80; G06V 10/82

(52) U.S. Cl. CPC G06V 20/49; G06V 10/7715; G06V 10/774; G06V 10/806; G06V 10/82; G06V 2201/031

(57) ABSTRACT

The present invention relates to a computer implemented method for video-based image segmentation. The method comprises obtaining an input video comprising multiple image frames, and generating predicted contour points of one or more target contours on one or more of the multiple image frames by a trained machine learning model. The model is trained with a training dataset prepared from multiple training videos, and each of the multiple training videos comprises multiple contiguous frames with annotated contour points of the one or more target contours. The architecture for this video-based image segmentation model may comprise a feature extractor, a feature fusion network, and a contour regressor, and the feature extractor may be constructed based on a Video Swin Transformer backbone. The video-based image segmentation method may be used to generate contours for echocardiogram images and may further be used to calculate clinical indicators to assess cardiac structure and function.

### RELATED APPLICATION

This application claims the benefit of the U.S. Provisional Application No. 63/744,827 filed on Jan. 13, 2025, titled “A VIDEO-BASED MULTI-CONTOUR LEARNING METHOD FOR CARDIAC IMAGE SEGMENTATION AND ANALYSIS,” which is incorporated herein by reference at its entirety.

### FIELD OF THE INVENTION

The present invention relates to a computer-implemented method for video-based image segmentation, especially for segmentation of echocardiogram videos.

### DESCRIPTION OF RELATED ART

Image segmentation is a computer vision process that divides a digital image into meaningful parts or segments to simplify analysis, identify objects, and locate boundaries. Traditional image segmentation techniques such as convolutional neural network (CNN)-based segmentation perform well in tasks like single image segmentation. Some traditional image segmentation techniques are also applied to image frames in videos. However, those models are usually inherently constrained by restricted receptive fields and a lack of temporal consistency due to their model architecture.

For example, cardiac ultrasound (echocardiography) is widely used in clinical practice to assess cardiac structure and function. However, because the heart's shape continuously changes throughout the cardiac cycle, relying solely on single-frame segmentation methods often fails to capture temporal consistency and shape stability. Existing technologies, such as single-frame mask segmentation or single-frame contour prediction, typically require additional post-processing steps to correct discontinuities and distortions in the segmentation results.

When the segmentation techniques are used to analyze echocardiogram videos, the predicted contours and their changes during cardiac cycles in different video frames of echocardiogram can further be used to calculate clinical indicators such as ejection fraction (EF) and global longitudinal strain (GLS) to assess cardiac structure and function. In echocardiography, tracking changes of ventricular volume requires identifying end systole (ES) and end diastole (ED) in each cardiac cycle. However, due to irregular temporal volume changes and signal noise, traditional methods such as peak finding and min/max finding methods frequently identify local maxima or minima instead of true extrema in one cardiac cycle, or require many predetermined parameters, which make those methods difficult to reliably estimate the cardiac cycle and ES/ED frames.

### SUMMARY OF THE INVENTION

This invention aims to overcome the limitations of single-frame approaches by introducing a video-based deep learning model that processes entire video rather than individual frames. By combining a contour annotation strategy with direct contour-point regression, it can learn and predict contour structures, thereby ensuring superior shape continuity and segmentation accuracy without the need for post-processing. Besides, a multi-contour annotation strategy may also be applied to enable the model to simultaneously learn and predict multiple contour structures (e.g., left ventricle endocardium, left ventricle epicardium, and left atrium endocardium in echocardiogram) to further enhance segmentation accuracy of the model.

As a video-based deep learning architecture, this invention treats the entire video as a continuous input rather than isolated frames. By leveraging local window self-attention (Video Transformer), it extracts both spatial and temporal information from all frames, capturing dynamic changes over time (e.g., during the full cardiac cycle). This video-based approach significantly improves resistance to frame-by-frame fluctuations and noise.

The present invention also provides a method for identifying locations of extrema in peaks and troughs in wave-like signals with fewer required parameters compared to prior art. Combining this method with predicted contours of echocardiogram, end systole (ES) and end diastole (ED) frames in each cardiac cycle can be accurately identified, and thus the changes of ventricular volume and strain can easily be tracked.

One aspect of the present disclosure provides a computer implemented method for video-based image segmentation. The method comprises steps of: (1) obtaining an input video comprising multiple image frames; and (2) generating predicted contour points of one or more target contours on one or more of the multiple image frames by a trained machine learning model. Here, the trained machine learning model is trained with a training dataset prepared from multiple training videos, and each of the multiple training videos comprises multiple contiguous frames with annotated contour points of the one or more target contours.

In one embodiment, the machine learning model comprises a feature extractor which applies local-window self-attention mechanism to both spatial and temporal dimensions to enhance segmentation stability across image frames. In preferred embodiments, the machine learning model may further comprise a multi-scale feature fusion mechanism to generate a fused feature map from outputs of different stages of the feature extractor; and the machine learning model may further comprise a contour regressor to generate the predicted contour points of the one or more target contours from the fused feature map. In one preferred embodiment, the feature extractor is constructed based on a Video Swin Transformer (VSwinT) backbone.

In one embodiment of the method, the annotated contour points of the one or more target contours are used as training ground truth during training. And in some embodiments, the machine learning model does not require mask-based segmentation or post-processing to generate the predicted contour points.

One application of the method as shown in preferred embodiments is video-based image segmentation for echocardiogram (cardiac ultrasound) videos. In those embodiments, the input video are echocardiogram videos.

In the cases where input videos are echocardiogram videos, the one or more target contours to be generated may comprise left ventricle (LV) endocardial line. The one or more target contours may further comprise left ventricle (LV) epicardial line and left atrium (LA) endocardial line.

The method for video-based image segmentation can be applied to echocardiogram videos with different views. For example, based on the settings and the training of the machine learning model, the input video may be an apical two-chamber view (A2C), an apical three-chamber view (A3C), or an apical four-chamber view (A4C) echocardiogram video.

The method for echocardiogram image frame segmentation may further comprise phase detection and/or strain calculation. The steps for phase detection may comprise: (1) for each of the plurality of the multiple image frames, calculating an left ventricular endocardium length from the predicted contour points of left ventricle (LV) endocardial line; and (2) identifying end-diastolic (ED) frames and end-systolic (ES) frames from the plurality of the multiple image frames based on the left ventricular endocardium length. After phase detection, strain calculation steps may also be performed. The steps for strain calculation may comprise: (1) obtaining one or more ED-ES pairs by pairing each of the identified ED frames with a following ES frame selected from the identified ES frames; (2) calculating a longitudinal stain value for each of the one or more ED-ES pairs; and (3) averaging the longitudinal stain values of the one or more ED-ES pairs to obtain a final left ventricular longitudinal strain (LVLS).

In one embodiment of phase detection, the end-diastolic (ED) frames and the end-systolic (ES) frames are identified by steps of: (1) obtaining an endocardium length series comprising the left ventricular endocardium lengths of the plurality of the multiple image frames; (2) obtaining an endocardium change series comprising differences of the left ventricular endocardium length between two consecutive image frames for each of the plurality of the multiple image frames; (3) based on the endocardium change series, determining a total state series which maximizes a penalized total path as an optimized state series; and (4) determining positions of the end-diastolic (ED) frames and the end-systolic (ES) frames based on the optimized state series. In this embodiment, the total state series comprises a state value describing systolic state or diastolic state at each time frame; and the total state series comprises a state value describing systolic state or diastolic state at each time frame, wherein a penalty value is introduced to the penalized total path every time when the state value changes in the total state series. The optimized state series may be determined by a dynamic programming algorithm.

In a preferred embodiment of identifying ED and ES frames, the end-diastolic (ED) frames are the image frames where the state changes from diastolic state to systolic state, and the end-systolic (ES) frames are the image frames where the state changes from systolic state to diastolic state in the optimized state series.

In a preferred embodiment of identifying ED and ES frames, said determining the optimized state series comprises iteratively calculating one or more penalized temporal paths and identifying one or more sub-optimal temporal paths from the one or more penalized temporal paths at each of the image frames.

Another aspect of the present disclosure provides a method to train a machine learning model concatenating a feature extractor, a multi-scale feature fusion mechanism and a contour regressor for generating contour points for video-based images. The method comprises: (1) obtaining a training dataset comprising multiple training data derived from multiple training videos, wherein each of the multiple training data comprises one of the multiple training videos as training input, and wherein each of the multiple training videos comprises a plurality of training image frames which are labeled with annotated contour points of one or more target contours as corresponding training ground truth; (2) inputting the training dataset into the machine learning model and performing parameter fitting to train the machine learning model, so that the machine learning model can use an input video comprising multiple image frames to generate predicted contour points of the one or more target contours for a plurality of the multiple image frames. In this method, the feature extractor applies local-window self-attention mechanism to both spatial and temporal dimensions of the input video, the multi-scale feature fusion mechanism receives outputs from different stages of the feature extractor generate a fused feature map based on the outputs, and the contour regressor receives the fused feature map and generates the predicted contour points of the one or more target contours for the plurality of the multiple image frames based on the fused feature map. In one embodiment, a mean squared error (MSE) loss function is used for parameter fitting of the machine learning model.

In one embodiment, the feature extractor is constructed based on a Video Swin Transformer (VSwinT) backbone. And in one embodiment, the feature extractor generates three temporally intact feature tensors for the multi-scale feature fusion mechanism to generate the fused feature map.

One application of the method is to train a machine learning model for echocardiogram image segmentation. In this application, each of the multiple training videos is an echocardiogram video, and the one or more target contours may comprise left ventricle (LV) endocardial line. The one or more target contours may further comprise left ventricle (LV) epicardial line and left atrium (LA) endocardial line.

Other objectives, advantages and novel features of the invention will become more apparent from the following detailed description when taken in conjunction with the accompanying drawings.

### BRIEF DESCRIPTION OF THE DRAWINGS

FIG. 1 illustrates the architecture for a video-based image segmentation model.

FIG. 2 illustrates the operations of two consecutive transformer blocks in the present disclosure.

FIGS. 3A-3C shows the contour prediction result of a VSwinT-based segmentation model. FIG. 3A is the generated contour points for an echocardiogram with apical four chamber (A4C) view; FIG. 3B is the generated contour points for an echocardiogram with apical three chamber (A3C) view; and FIG. 3C is the generated contour points for an echocardiogram with apical two chamber (A2C) view.

FIG. 4 is a state transition diagram illustrating ascending (S+) and descending (S−) states and possible transitions over time frames.

FIG. 5 shows an example of ED/ES detection with the dynamic programming method disclosed in the present application.

FIGS. 6A-6I illustrates scatterplots of LVGLS from 1050 individuals in a hybridization experiment. The x-axis represents strain measured by experts (%), and y-axis represents strain generated by automated measurement (%). The averaged strain values are provided as mean±SD; Spearman's p is reported to the fourth decimal place. The numbers in parentheses indicate the number of videos in the validation dataset (out of 1050) for which GLS could be successfully calculated by each hybridization combination. The hybrid model used in automatic measurements are: FIG. 6A: EchoNet-Dynamic; FIG. 6B: EchoNet+DP Strain; FIG. 6C: EchoNet+Unity Strain; FIG. 6D: VSwinT+EchoNet Strain; FIG. 6E: VSwinT-DP Strain; FIG. 6F: VSwinT+Unity Strain; FIG. 6G: Unity+EchoNet Strain; FIG. 6H: Unity+DP Strain; FIG. 6I: Unity-GLS.

FIGS. 7A-7I shows Bland Altman analysis between AI-strain and expert strain values. The x-axis represents mean strain of automated and expert's measurement (%), and γ-axis represents difference of strain (%), which is automated measurement minus expert's measurement. LOA: limits of agreement; n: the numbers in parentheses indicate the number of videos in the validation dataset (out of 1,050) for which GLS could be successfully calculated by each hybridization combination. The hybrid model used in automatic measurements are: FIG. 7A: EchoNet-Dynamic; FIG. 7B: EchoNet+DP Strain; FIG. 7C: EchoNet+Unity Strain; FIG. 7D: VSwinT+EchoNet Strain; FIG. 7E: VSwinT-DP Strain; FIG. 7F: VSwinT+Unity Strain; FIG. 7G: Unity+EchoNet Strain; FIG. 7H: Unity+DP Strain; FIG. 7I: Unity-GLS.

FIGS. 8A and 8B shows ROC analysis to predict the LV function by AI-strain with the proprietary combination. Among the 1050 validation videos, expert strain >−16% was defined as impaired LV function (FIG. 8A) and expert strain <−18% as normal LV function (FIG. 8B). ROC curves were used to evaluate the discriminative performance of the three proprietary combinations. AUC, area under the curve; LVGLS, left ventricular global longitudinal strain.

FIGS. 9A-9C illustrates performance of three AI models under poor image quality. FIGS. 9A and 9B shows AI model-generated contours for two selected frames of the echocardiogram video, and FIG. 9C shows the longitudinal strain calculated based on the generated contours of each frame. For EchoNet-Dynamic as shown in left panels of FIGS. 9A and 9B, the model first generates a mask (upper-left panels) and then converts it to the contour line (bottom-left panels). For Unity-GLS as shown in right panels of FIGS. 9A and 9B, the model first generates LV border heatmap (upper-right panels) and then converts it to the contour line (bottom-right panels).

FIGS. 10A-10C illustrates performance of three AI models under reverberation artifacts. FIGS. 10A and 10B shows AI model-generated contours for two selected frames of the echocardiogram video, and FIG. 10C shows the longitudinal strain calculated based on the generated contours of each frame.

FIGS. 11A-11F illustrates performance of three AI models under poor delineation of LV apex. FIGS. 11A-11E shows AI model-generated contours for five selected frames of the echocardiogram video, and FIG. 11F shows the longitudinal strain calculated based on the generated contours of each frame.

FIGS. 12A-12D shows an example with irrational end-diastolic or end-systolic time points due to a lack of temporal consistency. FIGS. 12A-12C shows AI model-generated contours for three selected frames of the echocardiogram video, and FIG. 12D shows the longitudinal strain calculated based on the generated contours of each frame.

### DETAILED DESCRIPTION OF PREFERRED EMBODIMENTS

The terminology used in the description presented below is intended to be interpreted in its broadest reasonable manner, even though it is used in conjunction with a detailed description of certain specific embodiments of the technology. Certain terms may even be emphasized below; however, any terminology intended to be interpreted in any restricted manner will be specifically defined as such in this Detailed Description section.

The embodiments introduced below can be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), graphics processing units (GPUs), etc.

### 1. Video-Based Image Segmentation

One aspect of the present application relates to a video-based deep learning model that processes image segmentation by using the information of entire video rather than the frame(s) undergoes segmentation tasks.

### Model Architecture

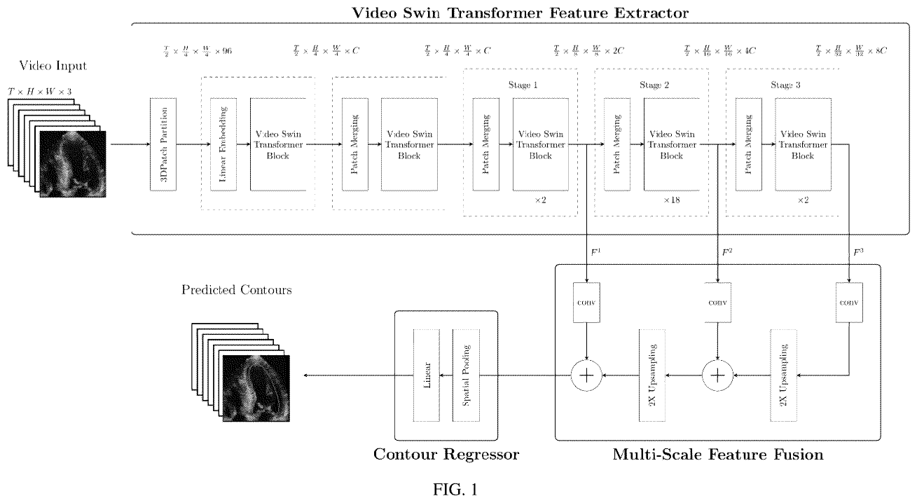

FIG. 1 illustrates the architecture for a video-based image segmentation model. The model comprises a feature extractor, a feature fusion network, and a contour regressor.

First the input video frames are fed into the feature extractor backbone, where multi-scale features are extracted from the intermediate layers of the backbone. A feature fusion network then merges the multi-scale features to generate a fused feature map. Lastly, the fused feature map is fed into the contour regressor to predict the contours of each frame.

In the implementation of the present application, this model architecture is used to construct a video-based image segmentation model for echocardiograms. Our segmentation model integrates a Video Swin Transformer (VSwinT) backbone for spatiotemporal feature extraction and a contour regression network for precise 2D contour point prediction of the left ventricular (LV) endocardium from echocardiogram videos. The architecture is carefully designed to leverage multi-scale representations and hierarchical attention mechanisms to process medical video data efficiently.

### Feature Extractor Backbone

The VSwinT backbone operates as the feature extractor, processing the echocardiogram video as 4 D tensors X∈R^{T*×H*×W*×C}, where T* is the frame number of the video, H* and W* are the height and width of each video frame, and C is the number of channels of each frame. The VSwinT backbone comprises three sequential stages (Stage 1-Stage3 as shown in FIG. 1).

The input video (4D tensor) is divided into non-overlapping 3D patches of size Pₜ×Pₕ×P_{w}, where Pₜ=2, Pₕ=8, and P_{w}=8, which are the patch size along temporal, height, and width dimensions, respectively.

Each patch is linearly embedded into a feature vector of dimension d, forming a sequence of tokens:
Z₀=Linear(X_{patches}))∈R^{T×H×W×d}, where

$$T = \frac{T}{{P}_{t}} ,$$

$$H = \frac{H}{{P}_{h}}$$

$$and$$

$$W = \frac{W}{{P}_{w}}$$

The Vision Transformers (ViT) utilize global self-attention (SA), which causes quadratic growth in computation and memory as the number of tokens increases. This scalability issue makes them inefficient for video tasks involving multiple frames. The Video Swin Transformer addresses this limitation by adopting a local window-based self-attention mechanism, similar to Swin Transformers, thereby enhancing efficiency for video-related applications.

Each Video Swin Transformer block is designed to process video tokens by integrating both spatial and temporal dimensions. Layer Normalization (LN) normalizes input features before both the 3D Windowed Multi-Head Self-Attention (3D W-MSA) and Feed-Forward Network (FFN) modules made by a 2-layer MLP, with GELU non-linearity. The 3D W-MSA performs self-attention within regular 3D windows, focusing on local token interactions. To enable cross-window connections and enhance representational capacity, 3D Shifted Window MSA (3D SW-MSA) is applied by shifting window configurations in consecutive blocks. FFN enhances the model's ability to learn complex representations, while residual connections after MSA/SW-MSA and FFN facilitate gradient flow and prevent vanishing gradients. This architecture effectively balances computational efficiency with robust spatiotemporal feature modeling, making the Video Swin Transformer ideal for tasks such as video classification and segmentation.

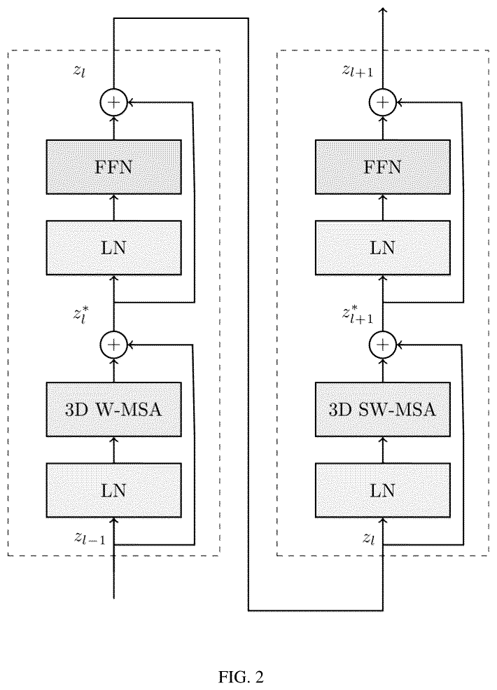

FIG. 2 illustrates the operations of two consecutive transformer blocks. The operations for two consecutive transformer blocks can be formally expressed as:

$${z}_{l}^{*} = 3 D W - MS A ( L N ( {z}_{l - 1} ) ) + {z}_{l - 1}$$

$${z}_{l} = F F N ( L N ( {z}_{l} ) ) + {z}_{l}^{*}$$

$${z}_{l + 1}^{*} = 3 D SW - MS A ( L N ( {z}_{l} ) ) + {z}_{l}$$

$${z}_{l + 1} = F F N ( L N ( {z}_{l + 1} ) ) + {z}_{l + 1}^{*}$$

where (1) zₗ₋₁ and zₗ denote the input and output features of the l-th transformer block, respectively; (2) 3D W-MSA is Multi-Head Self-Attention within regular 3D windows; (3) 3D SW-MSA is Shifted Window Multi-Head Self-Attention within shifted 3D windows; (4) LN is Layer Normalization; and (5) FFN is Feed-Forward Network.

To efficiently manage video data, the Video Swin Transformer employs a shifted window partitioning strategy, alternating between regular and shifted window configurations in consecutive transformer blocks. In regular window partitioning, the tokens are divided into non-overlapping 3D windows of size P×M×M, resulting in

$$⌈ \frac{T}{P} ⌉ \times ⌈ \frac{H}{M} ⌉ \times ⌈ \frac{W}{M} ⌉$$

windows, and Multi-Head Self-Attention (MSA) is applied within each 3D window independently. And in the subsequent transformer block, shifted window partitioning is applied, where the window partitioning is shifted by

$$( \frac{P}{2} , \frac{M}{2} , \frac{M}{2} )$$

tokens along the temporal, height, and window partitioning is shifted by width axes, respectively. Shifted Window MSA (SW-MSA) is applied here, allowing for cross-window connections while maintaining computational efficiency.

The self-attention operation within each window is defined as:

$$SA ( Q , K , V ) = SoftMax ( \frac{Q {K}^{⊤}}{\sqrt{d}} + B ) V ,$$

where:
(1) Q, K, V∈R^{d×N} are the query, key, and value matrices, respectively; (2) d is the dimension of the query and key vectors; (3) N=P×M×M is the number of tokens within a 3D window; and (4) B∈R^{N×N} represents the relative position bias matrix.

At each stage, neighboring patches are merged to reduce spatial resolution, preserving temporal length. For example, neighboring 2×2 spatial patches are merged, reducing tokens by a factor of 4. A linear layer unifies the channel dimension and doubles the token feature dimension. Spatial resolution (H, W) is halved at each stage.

This hierarchical approach balances efficiency and effective spatiotemporal modeling. The output of stage/is represented as:

$${Z}_{l} ∈ {R}^{T \times \frac{H}{\text{?}} \times \frac{W}{\text{?}} \times {d}_{l}}$$

where T is the constant temporal resolution, H, W are halved spatial dimensions, and dₗ is the token dimension, which doubles with each stage. This structure supports efficient and effective spatiotemporal modeling for tasks like video classification and segmentation.

Within each stage, a stack of Transformer blocks is applied. In the implementing example of the present application, every block first performs 3-D Windowed Multi-Head Self-Attention (3D W-MSA) over a local cuboid of 8×7×7 tokens, followed by a position-wise feed-forward network. The next block in the pair repeats the same operations after shifting the attention window by (4, 3, 3) tokens along the temporal, height, and width axes, respectively. This standard-window→shifted-window alternation allows tokens that were previously separated by the window boundary to attend to one another, thereby eliminating spatiotemporal seams and enlarging the receptive field linearly with network depth.

After the ending block of the last 3 stages, a spatial 2×2 patch-merging layer halves the height and width while preserving the full temporal length. The encoder therefore emits three temporally intact feature tensors that will later be fused: F¹=32 ×28×28, F²=32×14×14, F³=32×7×7.

### Feature Fusion Network

Outputs from three VSwinT stages (F¹, F², F³) are fused progressively, inspired by Fully Convolutional Networks (FCN)-style skip connections. Each feature map is first convolved to match channels, then upsampled and added to a higher-resolution map. This is because 3D W-MSA has already injected local temporal correlations, and shifted windows have propagated them across window borders. Then the fusion network can simply upsample (F², F³) to the resolution of F¹ and combine the three tensors with lightweight 2-D convolutions. The final fused feature map integrates both local details and global context.

Initially, the feature maps of the 3 stages have different resolutions. The smallest feature map F³ has height and width dimensions of (7,7); the intermediate feature map F² has dimensions (14,14); and the largest feature map F¹ has dimensions (28,28). Each feature map F^{l} undergoes a spatial refinement and channel unification using a 2D convolution operation. This step ensures that all feature maps have the same channel dimension D (here we choose D=1024), facilitating the subsequent upscaling and addition process: F^{l′}=Conv2D(F^{l}), F^{l′}∈R^{H}^{ₗ}^{×W}^{ₗ}^{×D}

The feature maps undergo progressive upscaling and addition. The smallest feature map F³′ is scaled up by a factor of 2 to match the resolution of F²:

$${F}_{s c a l e d}^{3 ′} = Upsample ( {F}^{3 ′} ) ;$$

the upscaled

$${F}_{s c a l e d}^{3}$$

is added to the intermediate, feature map F²′:

$${F}^{2 +} = {F}_{s c a l e d}^{3 ′} + {F}^{2 ′} ;$$

and the combined feature map F²⁺ is further scaled up by a factor of 2 to match the resolution of F¹′:

$${F}_{s c a l e d}^{2 +} = Upsample ( {F}^{2 +} ) .$$

### Finally,

$${F}_{scaled}^{2 ′ +}$$

is added to F^{1′}:

$${F}_{fused} = {F}_{s c a l e d}^{2 +} + {F}^{{1}^{′}} .$$

Then, the resulting fused feature map F_{fused} integrates information from all scales: F_{fused}∈R^{H}^{_{f}}^{×W}^{_{f}}^{×D}.

### Contour Regressor

The fused feature map Fₜ for each frame t is fed into the contour regression network, which predicts the 2D coordinates of 49 contour points representing the left ventricular (LV) endocardium.

The fused feature map for each frame is globally pooled into a compact vector zₜ: zₜ=GlobalPool(Fₜ)∈R^{D}.

The pooled feature vector, zₜ is directly passed through a fully connected linear layer to predict the 2D coordinates of the 49 contour points, resulting in total 49 coordinates (98 outputs total): Yₜ=Linear(zₜ)∈R⁹⁸.

The final predictions form a set of contours across all frames:

$${\{ {Y}_{t} \}}_{t = 1}^{T} .$$

### Training Objective

A Mean Squared Error (MSE) loss is used between predicted and ground-truth coordinates:

$$L = \frac{1}{T} {\sum }_{t = 1}^{T} \frac{1}{49} {\sum }_{i = 1}^{49} {‖ {y}_{t , i} - {\hat{y}}_{t , i} ‖}_{2}^{2} ,$$

where, y_{t,i} and ŷ_{t,i} are the ground-truth and predicted 2D coordinates for point i at frame t.

This model architecture can be used to train a machine learning model that can use an echocardiogram video as input to generate contour points of one or more structures (e.g., left ventricle endocardium, left ventricle epicardium, and/or left atrium endocardium). To achieve this goal, echocardiograms with annotated contour points were used as materials for model training.

### Training Materials and Model Training

2340 anonymized echocardiograms were used as raw materials of the training dataset, which includes 1282 videos from Taiwanese institutions, 72 videos featuring athletes from the Check-up Your Heart Program of the International University Sports Federation (FISU) during the 2017 Summer Universiade in Taipei, and 986 videos from the publicly available CAMUS dataset. These 2340 videos featured a diverse distribution of imaging systems, including Philips Medical Systems iE33 (38.12%) and EPIQ 7 C (16.41%), GE Vivid systems (2.68% collectively, FISU), and GE Vivid E95 machines (42.14%, CAMUS).

Similar to CAMUS annotation, a multitask annotation strategy was performed frame-by-frame on the 1282 institutional and 72 FISU videos by expert cardiologists who fulfill the Level III-IE competency criteria as defined by the American Society of Echocardiography. In addition to training data annotation, these same cardiologists also manually annotated and measured strain in a separate 1050-case validation dataset. While CAMUS annotated only the systolic phase, we performed full-length annotation of two cardiac cycles in each video. This included annotations of the LV endocardial line to define the inner boundary of the LV for assessing ventricular volume and function, the LV epicardial line to outline the outer boundary for measuring myocardial thickness and wall motion, and the LA endocardial line to mark the inner boundary of the LA for evaluating atrial size and function.

The pre-processing pipeline converts DICOM frames to 8-bit grayscale images and normalizes pixel values to a range of 0 to 255. Each frame is cropped to square dimensions based on the smaller of width and height, resized to 224×224 pixels, and expanded into three channels before being normalized to a range of 0 to 1. Videos are segmented into chunks of 64 frames with a 10-frame overlap, ensuring consistency in temporal resolution. For videos with fewer than 64 frames, the final frame is repeated to meet the required length. The resulting frames are stacked into tensors of shape [64, 224, 224, 3] and transposed to [3, 64, 224, 224] to align with the transformer's input specifications. All chunks are aggregated into tensors with a final shape of [#Chunk, 3, 64, 224, 224].

The model outputs the 2D coordinates of 49 LV endocardial contour points for each frame. This facilitates detailed tracking of left ventricular shape and function across the cardiac cycle, enabling precise contour-based assessments of myocardial performance.

Key training parameters were carefully selected to optimize performance. The loss function utilized was the mean squared error (MSE), calculated for each coordinate to minimize regression errors. The Adam optimizer was employed with a learning rate of 0.0001, a momentum of 0.9, and a weight decay of 0.0001, ensuring stable convergence during training. Extensive data augmentation techniques were implemented to improve the model's robustness and generalizability. These techniques included brightness adjustments with a variance of 75 and contrast scaling by a factor of 0.3. Additionally, geometric transformations were applied, such as shifts of up to 6.25%, scaling by up to 20%, and rotations within a range of +40°.

### Features of VSwinT-Based Segmentation Model

The constructed segmentation model utilizes continuous video frames to model the temporal progression of the heart's shape throughout the cardiac cycle, ensuring higher shape continuity and stability. When generating contours, it directly regresses contour points and thus eliminates the risk of fragmented or erroneous boundaries commonly encountered in mask-based systems. By producing final contour coordinates directly, the method obviates shape correction or smoothing steps typical of conventional approaches.

Besides, during model training we performed a multi-contour annotation approach which includes LV endocardial line (inner boundary of the left ventricle), LV epicardial line (outer boundary of the left ventricle) and LA endocardial line (inner boundary of the left atrium). The model can therefore learn to generate different contours, which expedite clinical analysis by providing multiple structures in one pass. Moreover, the method concurrently learns and predicts the endocardial and epicardial contours of the left ventricle, as well as the endocardial contour of the left atrium, which may enhance segmentation accuracy across various cardiac phases and patient groups.

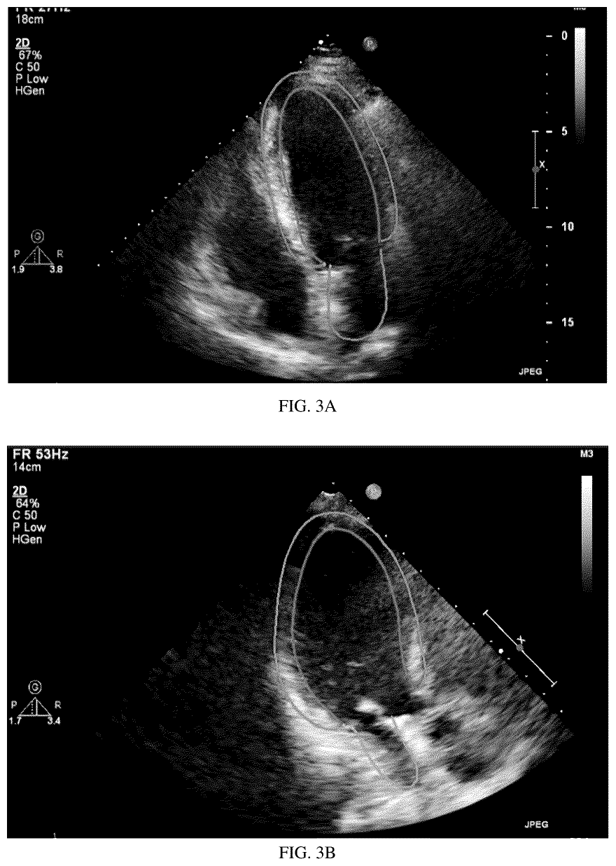

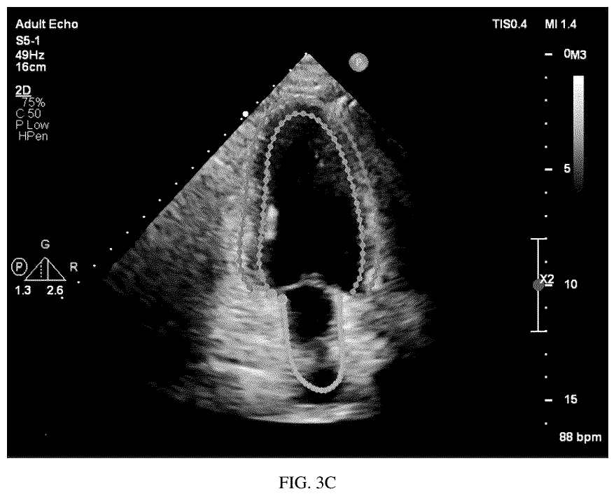

FIGS. 3A-3C shows the contour prediction result of a VSwinT-based segmentation model. By selecting different views of echocardiograms as training input, the model can generate contour points for image frames with corresponding views. FIG. 3A is the generated contour points for an echocardiogram with apical four chamber (A4C) view; FIG. 3B is the generated contour points for an echocardiogram with apical three chamber (A3C) view; and FIG. 3C is the generated contour points for an echocardiogram with apical two chamber (A2C) view.

### Comparison of VSwinT-Based Model and Other Segmentation Models

Table 1 shows the computational and resource profiles of our VSwinT model and two other frame-based segmentation models, Unity and EchoNet. Unity model utilizes HRNet to directly predict key points along the LV contour, and EchoNet directly estimates the left ventricular (LV) mask from individual frames. Among them, VSwinT excels in handling video input with remarkable efficiency and scalability.

TABLE 1
Comparison of Unity, EchNet and VSwinT models

| | | | | | | GPU |
|---|---|---|---|---|---|---|
| | | | | | #param- | memory |
| Model | Frame | Input | | GFLOPS | eters | usage |
| name | num | resolution | GFLOPS | per frame | (M) | (MB) |
| Unity | 1 | 608 × 608 | 275.77 | 275.77 | 66.97 | 499.85 |
| EchoNet | 1 | 112 × 112 | 7.83 | 7.83 | 39.63 | 162.22 |
| VSwinT | 64 | 224 × 224 | 574.53 | 8.98 | 121.38 | 2289.51 |

It shows from Table 1 that VSwinT processes 64 frames at 224×224 resolution with a total cost of 574.53 GFLOPs, translating to just 8.98 GFLOPs per frame. This efficiency highlights its ability to handle large temporal data without excessive per-frame overhead, making it ideal for video-based tasks. With 121.38M parameters, VSwinT outperforms in feature extraction and temporal modeling, offering rich representational power suited for complex video understanding tasks. Despite processing 64 frames simultaneously, VSwinT's GPU memory usage (2289.51 MB) remains proportional to its computational needs, showcasing effective resource utilization for high-throughput video pipelines.

Unity's high computational cost (275.77 GFLOPs per frame) reflects its focus on detailed spatial processing (608×608) which limits scalability. EchoNet is lightweight, with 7.83 GFLOPs per frame and 162.22 MB memory usage, designed for low-resolution, resource-constrained tasks. However, its analysis ability is restricted to single image frame.

In summary, VSwinT stands out as the most balanced and efficient model for video applications, combining low per-frame computational cost, high feature capacity, and scalability for multi-frame processing. It offers a compelling choice for video tasks requiring temporal and spatial modeling at scale.

### 2. ED/ES Identification

The lengths of predicted LV endocardium contour in different image frames form a length versus time (frame) series with a wave-like appearance. The end-diastole (ED) frames and end-systole (ES) frames can be detected with a computer-implemented method identifying extremum locations of wave signals.

In this method, an objective function derived from a wave signal is used for extremum location identification. The objective function has a “state” parameter related to the different states (e.g., ascending and descending) of the wave signal at each time frame. By choosing suitable state parameters which maximize the objective function and identifying the locations where the state parameter changes in the wave signal, extremum locations (i.e., turning points) and the corresponding signal values of the wave signal can then be identified.

### Objective Function

The goal of the objective function is to ensure identification of state transitions while maximizing the difference of signal value between an identified peak and its neighboring trough. To achieve this goal, a transition penalty is introduced to the objective function to prevent local extrema caused by noise from being incorrectly identified as true extrema in a wave period. The objective function resembles the cumulated path an imaginary point has traveled during signal recordation, except that a penalty value is subtracted from the cumulated path every time a state transition occurs. Because the objective value is penalized every time when a transition occurs, false identification of noise-derived local extrema as turning points is suppressed by the transition penalty.

In a preferred embodiment, the objective function has the form:

$$Objective = {\sum }_{t = 1}^{T} ( {V}_{diff} ( t ) \cdot s ( t ) - Z ( t ) ) . \qquad ( Eq . 1 )$$

In this function, V_{diff}(t)=V(t)−V(t−1), which is the difference of signal value between consecutive frames t−1 and t, or the current displacement between time frames t−1 and t. The time frame is counted from the first frame (t=1) to the last frame (t=T). s(t) describes the state in time frame t. If the wave signal in time frame t is in ascending state (i.e., the signal value goes up), s(t)=1; and if the wave signal is in descending state (i.e., the signal value goes down), s(t)=−1. Z(t) is the transition penalty in time frame t. A penalty value σ is applied when switching between ascending and descending states to ensure that the algorithm avoids unnecessary state transitions. That is, Z(t)=0, if s(t)=s(t−1), and Z(t)=σ, if s(t)≠s(t−1). The objective function may be regarded as a penalized total path, which is calculated by summing up the penalized individual path V_{diff}(t)·s(t)−Z(t) of all time frames. And for calculating the penalized individual path at t=1, V(0) is defined as 0 for both s(0)=1 and s(0)=−1.

### Penalty Value

Penalty value (σ) may be any appropriate value to avoid unnecessary state transitions. In a preferred embodiment, a statistical measure which measures the spread or dispersion of the signal value is used. Examples of statistical measures which can be used as the penalty value include but not limited to interquartile range (IQR), standard deviation (SD), average absolute deviation (AAD), or median absolute deviation (MAD) of the wave signal. The definitions and key features of those statistical measures are described below.

### 1. Interquartile Range (IQR)

The definition of IQR is the range of the middle 50% of the data, i.e., the difference between the third quartile (Q3) and the first quartile (Q1), which has a formula: IQR=Q3−Q1. IQR is robust to outliers, and it focuses on the central portion of the data. However, it will ignore data in the tails.

### 2. Standard Deviation (SD)

The definition of SD is the square root of variance, measuring the average deviation from the mean in the same units as the data, which has a formula SD=\sqrt{Variance}. It has the same units as the data and is widely used. However, it is more sensitive to outliers.

### 3. Average Absolute Deviation (AAD)

The definition of AAD is the average of the absolute deviations from the mean, which has a formula

$$AAD = \frac{1}{n} {\sum }_{i = 1}^{n} \vert {x}_{i} - \overline{x} \vert .$$

Compared to variance or standard deviation, it is less sensitive to outliers than variance, but it is less common in statistical software and textbooks.

### 4. Median Absolute Deviation (MAD)

Median Absolute Deviation (MAD) is a robust measure, which is the median of the absolute deviations from the median of the data, and has a formula of MAD=median (|xᵢ−median(x)|). It is Highly robust to outliers, but it may not be sensitive enough for datasets with fine variability.

### State Determination

For a given signal V, the aim is to find a state series s(t) (for t=1 to T) which maximizes the objective value of the objective function

$${\sum }_{t = 1}^{T} ( {V}_{diff} ( t ) \cdot s ( t ) - Z ( t ) ) .$$

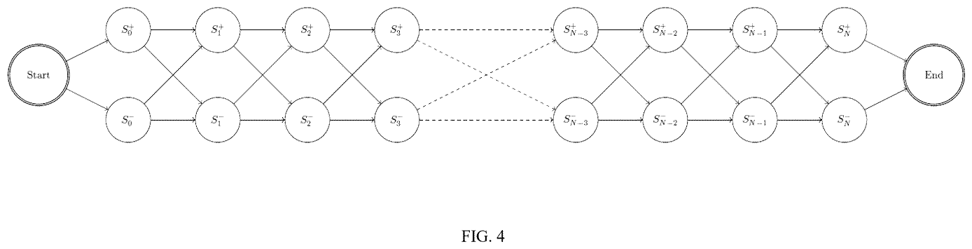

The maximal objective value is called an optimized final path. In each frame, the state s is either in ascending state (+1) or descending state (−1). The diagram shown in FIG. 4 illustrates possible state transitions of a wave signal. In short, in each time frame, the state s may be with the value +1 or −1.

If the state series

$${\{ s ( t ) \}}_{t = 1}^{T}$$

is determined, the objective value can be calculated by simply adding up penalized individual path P(t)=V_{diff}(t)·s(t)−Z(t) of all time frames. Since V_{diff}(t)=V(t)−V(t−1), and the value of Z(t) is determined by s(t) and s(t−1), it can be easily concluded that calculating the value of penalized individual path of the current time frame t only requires the information of V(t), V(t−1), s(t) and s(t−1). And because V(t) and V(t−1) are given values, and both s(t) and s(t−1) have only two possible values (+1 or −1), it can be concluded that the penalized individual path P(t) can only have four possible values, which are V_{diff}(t), V_{diff}(t)−0, −V_{diff}(t), and −V_{diff}(t)−σ.

Now we define a penalized temporal path:

$$T P ( {t}^{′} , {s}^{′} ) = {\sum }_{t = 1}^{t ′} ( {V}_{diff} ( t ) \cdot s ( t ) - Z ( t ) ) ,$$

wherein s(t′)=s′. (Eq. 2)
The penalized temporal path is the penalized path calculated from t=1 to t=t′ with a state value s′ at time frame t′. If t′ is the time frame we currently focus on, then s′ is the current state value. Among all possible TP(t′, s′), the maximum TP value with a current state value s′ at current time frame t′ is called current path and denoted as TP_{Max}(t′, s′) or DP(t′, s′). The current path with current state s′=1 is called first current path, and the other one with current state s′=−1 is called second current path.

By definition, the penalized temporal path of the current time frame t equals the penalized temporal path of the previous time frame t−1 (which is called previous temporal path) plus the penalized individual path of the current time frame t. That is, TP(t−1, s′)+V_{diff}(t)·s(t)−Z(t)=TP(t, s). Here, s′ is the state value of previous time frame t−1, which is called previous state value. We define the maximum TP value of previous time frame TP_{Max}(t−1, s′) as previous path, wherein the one with previous state s′=1 is called first previous path, and the other one with previous state s′=−1 is called second previous path. In other words, the first previous path is the largest among the previous temporal path having a status of ascending at the previous time frame t−1, and the second previous path is the largest among the previous temporal path having a status of descending at the previous time frame t−1. With the above equation, we have a recurrence relation:

$$T {P}_{Max} ( t , s ) = {max}_{{s}^{′} ∈ \{ 1 , 1 \}} [ {TP}_{Max} ( t - 1 , {s}^{′} ) + {V}_{diff} ( t ) \cdot s - Z ( t ) ] . \qquad ( Eq . 3 )$$

This means that TP_{Max}(t, 1) is the larger one of first ascending path TP_{Max}(t−1, 1)+V_{diff}(t) and second ascending path TP_{Max}(t−1, −1)+V_{diff}(t)−σ. Similarly, TP_{Max}(t, −1) is the larger one of first descending path TP_{Max}(t−1, 1)−V_{diff}(t)−σ and second descending path TP_{Max}(t−1, −1)−V_{diff}(t). Using the recurrence relation of Eq. 3, TP_{Max}(t, 1) and TP_{Max}(t, −1) can further be used to calculate TP_{Max}(t+1, 1) and TP_{Max}(t+1, −1).

Based on the above analysis, it shows that TP_{Max}(t, s) for all t can be calculated, including TP_{Max}(T, s). And since the optimized final path is the largest among all possible objective values, it can be determined by selecting the larger one of TP_{Max}(T, 1) and TP_{Max}(T, −1).

It is known by Eq. 3 that each TP_{Max}(t, s) corresponds to a TP_{Max}(t−1, s′) selected from TP_{Max}(t−1, 1) and TP_{Max}(t−1, −1). Therefore, if we record the values of first previous path TP_{Max}(t−1, 1) and second previous path TP_{Max}(t−1, −1), we can get the previous state value s′ at time frame t−1 which yields TP_{Max}(t, s). In other words, after calculating TP_{Max}(t, s), two previous state values can be determined, which are the previous state value corresponding to TP_{Max}(t, 1), and the previous state value corresponding to TP_{Max}(t, −1). Starting from the optimized final path and its corresponding state value s (T), we can trace back previous state values all along to t=1, and the resulting state series

$${\{ s ( t ) \}}_{t = 1}^{T}$$

is the optimized state series, which maximize the objective value.

The optimized state series may also be obtained via an alternative approach. Based on Eq. 3, it is known that each current path TP_{Max}(t, s) corresponds to a previous path TP_{Max}(t−1, s′) selected from the first previous path TP_{Max}(t−1, 1) and the second previous path TP_{Max}(t−1, −1). Therefore, the state series may be recorded and updated sequentially from the first time frame to the last time frame. In this approach, the calculated states of previous time frames are recorded as a first previous state series and a second previous state series, and the first previous state series and the second previous state series are updated with newly calculated results in every time frame. In particular, the first previous state series may be a state series

$${\{ s \}}_{1}^{t - 1}$$

corresponding to the first previous path TP_{Max}(t−1, 1), and the second previous state series may be a state series

$${\{ s \}}_{1}^{t - 1}$$

corresponding to the second previous path TP_{Max}(t−1, −1).

The optimized state series may be determined as follows. In each time frame, the first previous path TP_{Max}(t−1, 1), the first previous state series, the second previous path TP_{Max}(t−1, −1), the second previous state series, and the current displacement V_{diff}(t) are obtained. Then the first current path TP_{Max}(t, 1) and the second current path TP_{Max}(t, −1) are calculated based on the first previous path, the second previous path, and the current displacement. As described above, this may be done by selecting the larger one of the first ascending path TP_{Max}(t−1, 1)+V_{diff}(t) and the second ascending path TP_{Max}(t−1, −1)+V_{diff}(t)−σ as the first current path TP_{Max}(t, 1), and selecting the larger one of the first descending path TP_{Max}(t−1, 1)−V_{diff} (t)−σ and the second descending path TP_{Max}(t−1, −1)−V_{diff}(t) as the second current path TP_{Max}(t, −1). Next, the first previous path and the second previous path are respectively replaced by the first current path and the second current path for use in the next iteration. Lastly, based on the selections which yield the first and second current paths, a first current state series corresponding to the first current path and a second current state series corresponding to the second current path can be obtained. For example, if the first current path is the first ascending path, then an ascending state is added to the first previous state series as the first current state series; if the first current path is the second ascending path, then an ascending state is added to the second previous state series as the first current state series; if the second current path is the first descending path, then a descending state is added to the first previous state series as the second current state series; and if the second current path is the second descending path, then a descending state is added to the second previous state series as the second current state series. Then, the first previous state series and the second previous state series are also respectively replaced by the first current state series and the second current state series for use in the next iteration.

The above steps are repeated until the last time frame. Then, the larger of the first current path and the second current path at the last time frame is selected as an optimized final path, and the total state series corresponding to the optimized final path is selected as the optimized state series.

### Dynamic Programming

A dynamic programming (DP) algorithm can be used to perform the above objective value calculation and state series determination tasks. As described above,

$$T {P}_{Max} ( t , s ) = {max}_{{s}^{′} ∈ \{ 1 , 1 \}} [ {TP}_{Max} ( t - 1 , {s}^{′} ) + {V}_{diff} ( t ) \cdot s - Z ( t ) ] ,$$

wherein Z(t)=0, if s(t)=s(t−1), and Z(t)=σ, if s(t) s(t−1). To calculate the value of TP(1, s), TP_{Max}(0, s′) is set to 0 for both s′=1 and s′=−1.

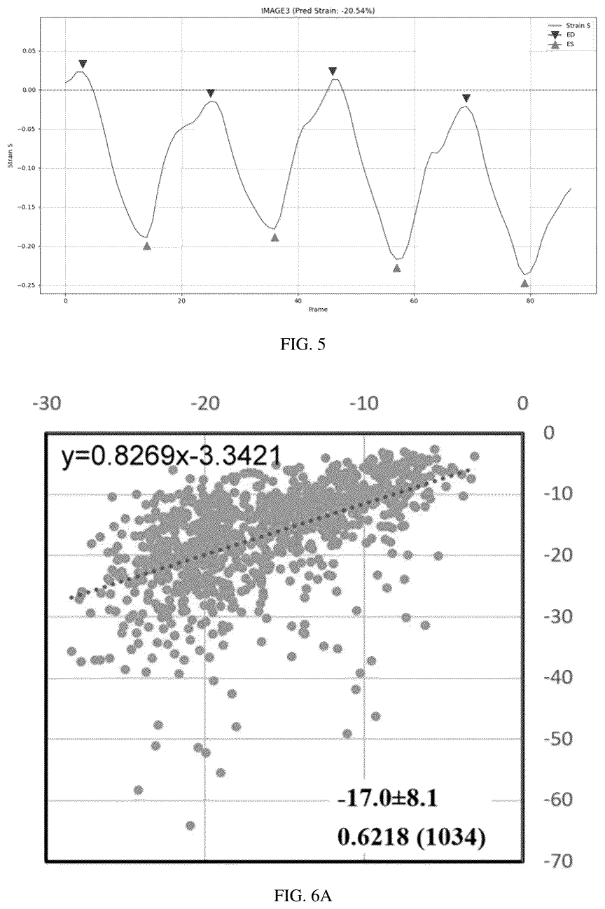

In our model, the ED/ES detection algorithm leverages dynamic programming to detect diastolic and systolic transitions optimally. FIG. 5 shows an example of ED/ES detection with the disclosed dynamic programming method. The curve shows Lagrangian Strain (which will be described below) values for different frames, and the ED and ES frames tracked by the DP algorithm. The curve is the strain signal, and the tracked ED and ES frames are indicated by the marks, ▾ and ▴. This example shows that our model can ensure accurate phase detection by maximizing the total objective function.

### 3. Strain Calculation

Following the extraction of the LV contour from the model, the left ventricular longitudinal strain (LVLS) can be calculated from the length of the left ventricular contours of all video frames. The is calculated by the Lagrangian Strain formula

$${S}_{L} ( t ) = \frac{L ( t ) - {L}_{0}}{{L}_{0}} ,$$

where: L(t) is the endocardial contour length at time t; and Lo is the reference length, which is the maximum endocardial contour length for that cycle. LVLS is the stain where L(t) is the endocardial contour length in end-systole frame with a minimum endocardial contour length.

LV segmentation models are commonly paired with proprietary strain calculation algorithms for AI-based LVGLS calculations. To explore the interaction between LV contour prediction and strain calculation algorithms, a hybrid experimental setup was designed by combining three segmentation models with three strain calculation algorithms, resulting in nine distinct configurations. The three strain estimation methods are (1) DP Strain (our model), (2) EchoNet Strain Calculation and (3) Unity Strain Calculation.

DP Strain Model utilizes dynamic programming to identify the global optimum ED and ES phases without requiring parameter tuning, enhancing robustness against noise.

EchoNet Strain Calculation involves estimating the LV endocardial mask with EchoNet, dilating it, extracting the LV endocardial contour, obtaining the length time series, identifying local maxima and minima using SciPy's find _peaks function (which requires parameter tuning to optimize performance), pairing ED and ES points, and averaging the strain over these pairs.

Unity Strain Calculation acquires the LV endocardial heatmap from the Unity model, converts it to the LV endocardial contour, obtains the length time series, identifies local maxima and minima using SciPy's find _peaks (requiring parameter tuning), averages the ED lengths, averages the ES lengths, and uses these averages to estimate strain.

### 4. Performance Evaluation

The validation phase assesses the performance and reliability of our model's segmentation results and strain estimation using a comprehensive set of echocardiogram videos. Three contour prediction methods and three strain measurement methods are included in the evaluation.

The three segmentation models for comparison are our VSwinT-based model, EchNet model, and Unity model, as described above in Section 1. The three strain estimation methods employed are our DP-Strain model, EchoNet Strain Calculation model, and Unity Strain Calculation model, as described above in Section 3.

The mixed analysis evaluates the interaction between contour prediction and strain measurement by comparing the performance across the nine combinations. This approach assesses how segmentation accuracy influences strain estimation reliability, ensuring that the segmentation models not only perform well independently but also contribute effectively to accurate downstream clinical assessments.

### Evaluation Dataset

Two distinct test sets are utilized here: (1) Primary Evaluation Set, and (2) HMC-QU Evaluation Set.

The Primary Evaluation Set is a dataset of 1,050 Apical Four Chamber (A4C) echocardiograms with manual longitudinal strain measurements and LV endocardial contour labels at end-diastole and end-systole as contour and strain annotations. This evaluation set is used to evaluates segmentation performance and strain estimation accuracy.

The HMC-QU Evaluation Set is a dataset of 162 Apical Four Chamber (A4C) videos from HMC-QU open dataset (publicly available). This dataset includes multiple pathological conditions and imaging scenarios, enabling the evaluation of the model's clinical applicability. No ground truth strain values are provided in this evaluation set, and this set is used for pairwise comparison of strain performance across different models.

### Models for Performance Comparison

Our VSwinT-based model and DP-Strain model can be integrated (the integrated model is abbreviated as VSwinT-DP Strain) to perform strain calculated directly from an input echocardiogram. EchoNet-Dynamic and Unity-GLS are two other integrated models used for performance comparison for both contour prediction and strain measurement. These two models are frame-based segmentation models commonly used for baseline comparison in echocardiographic analysis.

EchoNet-Dynamic is a deep learning model for assessing cardiac function in echocardiogram videos. It directly estimates the left ventricular (LV) mask from individual frames with EchNet model, followed by computer vision algorithms to extract the LV contour from the mask.

Unity-GLS utilizes HRNet to directly predict key points along the LV contour with Unity model, providing precise contour delineation.

Both models operate on single frames without incorporating temporal context, making them suitable for static frame evaluations but limited in capturing dynamic cardiac motion. They are selected to demonstrate that our video-based model can perform better than those frame-based models.

Besides three proprietary models (VSwinT-DP Strain, EchoNet-Dynamic and Unity-GLS), six hybrid models that combining different LV segmentation models with different strain measurement models are also tested for model performance comparison. The six hybrid models tested were VSwinT+EchoNet Strain, VSwinT+Unity Strain, EchoNet+DP Strain, EchoNet+Unity Strain, Unity+DP Strain, and Unity+EchoNet Strain.

### Process Completeness

Process completeness assesses each model's ability to execute the entire segmentation and strain analysis pipeline without failures. This evaluation includes successful contour segmentations and strain values successfully estimated. Successful contour segmentations measures the number of videos for which contour segmentation was successfully completed; and strain values successfully estimated measures The number of strain measurements successfully derived from the segmentation results. This metric shows the robustness of the segmentation and strain estimation processes across the dataset.

### Performance Metrics

Two performance metrics, segmentation performance metrics and strain estimation performance metrics, are independently used to evaluate segmentation performance and strain estimation performance of different models. Notably, although our VSwinT-based architecture can be trained to generate different contours (e.g., LV endocardium, LV epicardium, and LA endocardium) with echocardiograms of different views (e.g., A4C, A3C, and A2C), this evaluation mainly focuses on LV endocardium segmentation in A4C view due to the limitations of other comparing methods in this evaluation.

### Segmentation Performance Metrics

Segmentation performance is evaluated using the following metrics across 2,100 annotated frames (1,050 videos at end-diastole and end-systole phases). Four indicators, (1) Dice Coefficient, (2) Intersection over Union (IoU), (3) Hausdorff Distance, and (4) Average Hausdorff Distance are applied for evaluation.

Dice Coefficient measures the overlap between predicted and ground truth contours; Intersection over Union (IoU) calculates the ratio of the intersection to the union of predicted and ground truth areas; Hausdorff Distance determines the maximum distance between points on predicted and ground truth contours; and Average Hausdorff Distance provides an average measure of the distance between predicted and ground truth contours. These metrics offer a comprehensive assessment of each model's contour prediction accuracy.

The contour segmentation performance of different models is shown in Table 2 below.

TABLE 2
Segmentation performance of EchoNet-Dynamic model, VSwinT-DP Strain model and Unity-GLS model

| | Dice | IoU | Hausdorff | Avg Hausdorff | Success Count |
|---|---|---|---|---|---|
| Contour Model | (Mean ± Std) | (Mean ± Std) | (Mean ± Std) | (Mean ± Std) | (total 2100) |
| Echonet- | 0.8307 ± 0.0799 | 0.7172 ± 0.1011 | 37.5010 ± 17.8117 | 11.2053 ± 6.6333 | 2068 |
| Dynamic | | | | | |
| VSwinT-DP | 0.9073 ± 0.0523 | 0.8342 ± 0.0800 | 17.1316 ± 7.4844 | 7.1883 ± 3.9248 | 2100 |
| Strain | | | | | |
| Unity-GLS | 0.9097 ± 0.0774 | 0.8410 ± 0.0969 | 19.3002 ± 26.0130 | 7.5315 ± 6.0076 | 2085 |

The above table compares the performance of three contour segmentation models-EchoNet-Dynamic, Our VSwinT-DP Strain, and Unity-GLS-across multiple metrics for evaluating contour accuracy and reliability. The results show that our model achieves high Dice and IoU values, comparable to Unity-GLS, while significantly outperforming in Hausdorff metrics, indicating better shape preservation and reduced outliers. Additionally, our model achieves a perfect success count, surpassing both EchoNet-Dynamic and Unity-GLS in reliability.

### Strain Estimation Performance Metrics

Following the extraction of the LV contour from the model, the Left Ventricular Longitudinal Strain (LVLS) can be calculated from the length of the left ventricular contours of all video frames. Four metrics, (1) Mean Squared Error (MSE), (2) Root Mean Squared Error (RMSE), (3) R-Squared (R²), and (4) Pearson's Correlation Coefficient (rrr), are employed to evaluate the strain estimation performance.

Mean Squared Error (MSE) and Root Mean Squared Error (RMSE) quantify the deviation between predicted and ground truth strain values; R-Squared (R²) indicates the proportion of variance in the ground truth strain explained by the predicted values; and Pearson's Correlation Coefficient (rrr) measures the linear correlation between predicted and ground truth strain values, reflecting the consistency of the estimation. These metrics provide a comprehensive assessment of the accuracy and reliability of the model's strain estimation capabilities.

The strain estimation performance of different models is shown in Table 3 below.

TABLE 3
Strain estimation performance of EchoNet-Dynamic
model, VSwinT-DP Strain model and Unity-GLS model

| | Strain Success | | | | |
|---|---|---|---|---|---|
| Contour | Count (total | | | | Correlation |
| Model | 1050) | MSE | RMSE | R² | Coeff |
| EchoNet- | 1034 | 47.1412 | 6.8659 | −0.6498 | 0.5458 |
| Dynamic | | | | | |
| VSwinT-DP | 1050 | 7.3476 | 2.7106 | 0.7410 | 0.8846 |
| Strain | | | | | |
| Unity-GLS | 1007 | 24.9325 | 4.9932 | 0.1225 | 0.6408 |

The table compares the performance of three models—EchoNet-Dynamic, Our VSwinT-DP Strain, and Unity-GLS—in estimating Left Ventricular Longitudinal Strain (LVLS). The results highlight that our model achieves a perfect strain success count (1050/1050) and outperforms others with the lowest MSE (7.3476) and RMSE (2.7106), alongside the highest R² (0.7410) and correlation coefficient (0.8846). These metrics demonstrate its superior accuracy, consistency, and reliability in strain estimation compared to EchoNet-Dynamic and Unity-GLS.

### Hybridization of Segmentation Model and Strain Calculation Algorithm

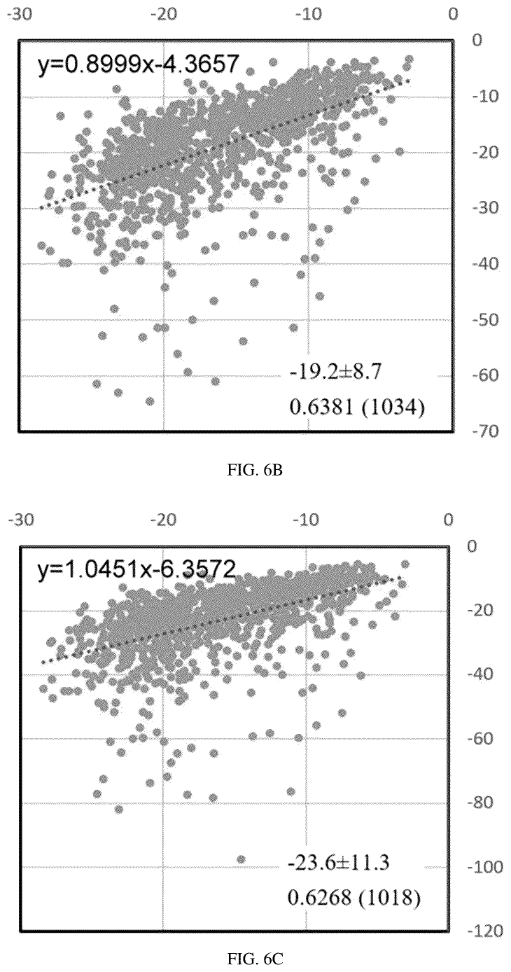

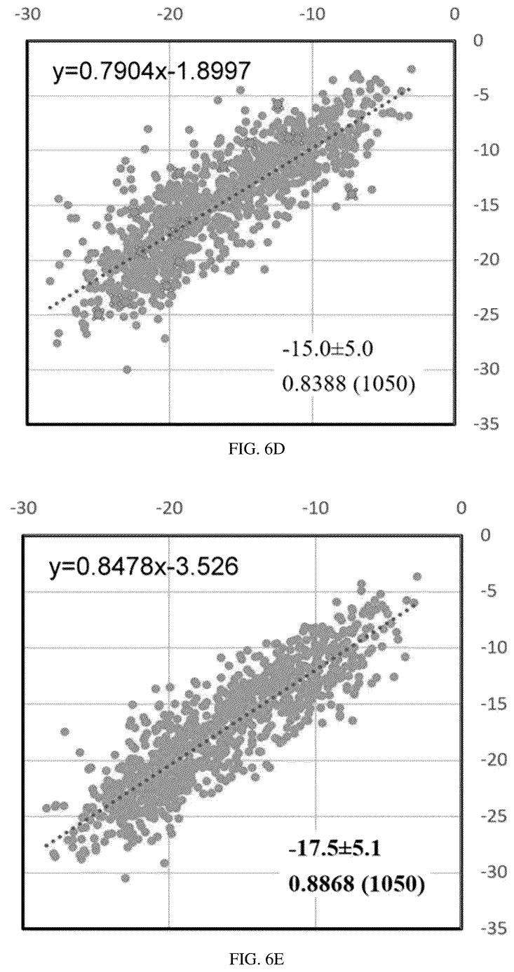

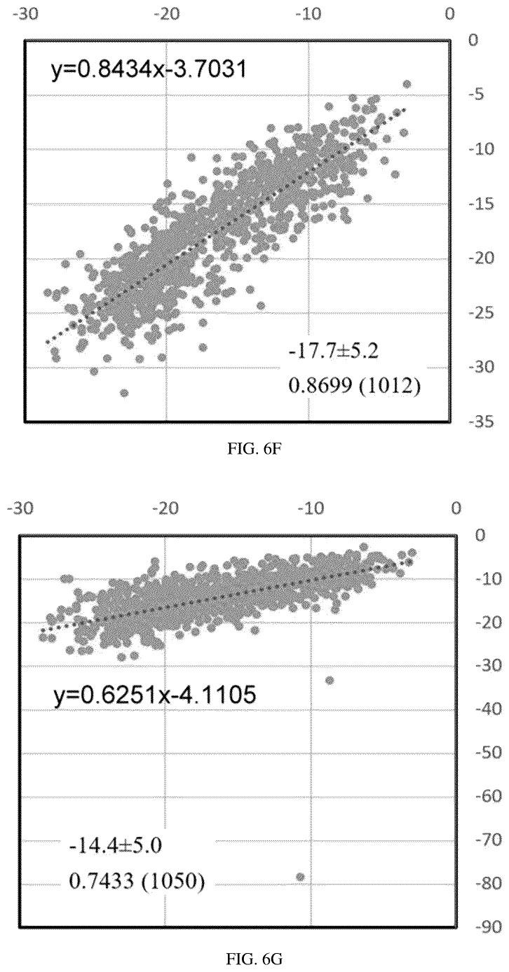

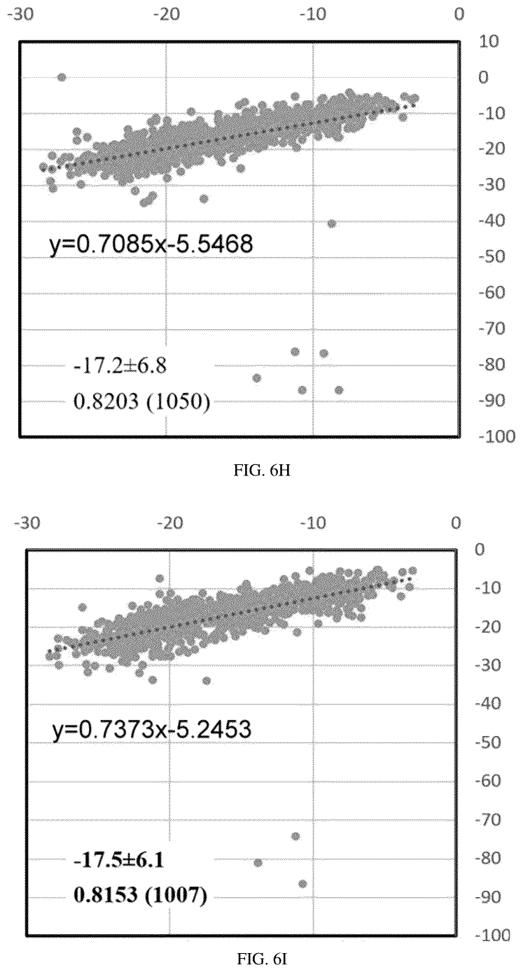

Besides strain estimation performance of the three proprietary models (VSwinT-DP Strain, EchoNet-Dynamic and Unity-GLS), six hybrid models that combining different LV segmentation models with different strain measurement models are also tested. Nine hybridized strain methods in total were tested with a testing dataset, and the results were compared with expert-derived strain values. The results are plotted as 9 scatterplots of AI-derived strain values against expert-derived strain values, as shown in FIGS. 6A-6I. FIGS. 6A-6I summarizes Spearman's r value of nine hybridized strain methods comparing to expert strain values. VSwinT-DP Strain had the highest correlation with expert-derived strain. The intraclass correlation coefficient (ICC) between three proprietary combinations and expert strain values were VSwinT+DP Strain: 0.868 (0.807-0.905), p<0.001; Unity-GLS: 0.627 (0.583-0.667), p<0.001; and EchoNet-Dynamic: 0.501 (0.454-0.545), p<0.001.

Compared with proprietary combination, the EchoNet-Dynamic contour will cause a higher absolute strain value (EchoNet+DP Strain vs. VSwinT-DP Strain, n=1034: −19.18±9.72 vs. −17.51±5.13, p<0.001; EchoNet+Unity Strain vs. Unity-GLS, n=979: −23.52±11.12 vs. −17.31±8.86, p<0.001). Interestingly, the EchoNet-Dynamic strain algorithm will cause a lower absolute strain value (VSwinT-DP Strain vs. VSwinT+EchoNet Strain, n=1050: −17.53±5.11 vs. −14.95=5.04, p<0.001; Unity-GLS vs. Unity+EchoNet Strain, n=1007: −17.46±6.14 vs. −14.43±5.04, p<0.001). On the other hand, either the change of segmentation contour or the strain calculation algorithm for VSwinT-DP Strain and Unity-GLS with the other did not cause remarkable strain value deviation (VSwinT-DP Strain vs. Unity+DP Strain, n=1050: −17.52=5.11 vs. −17.24=6.83, p=0.1151; VSwinT-DP Strain vs. VSwinT+Unity Strain, n=1012: −17.585.10 vs. −17.665.19, p=0.0607).

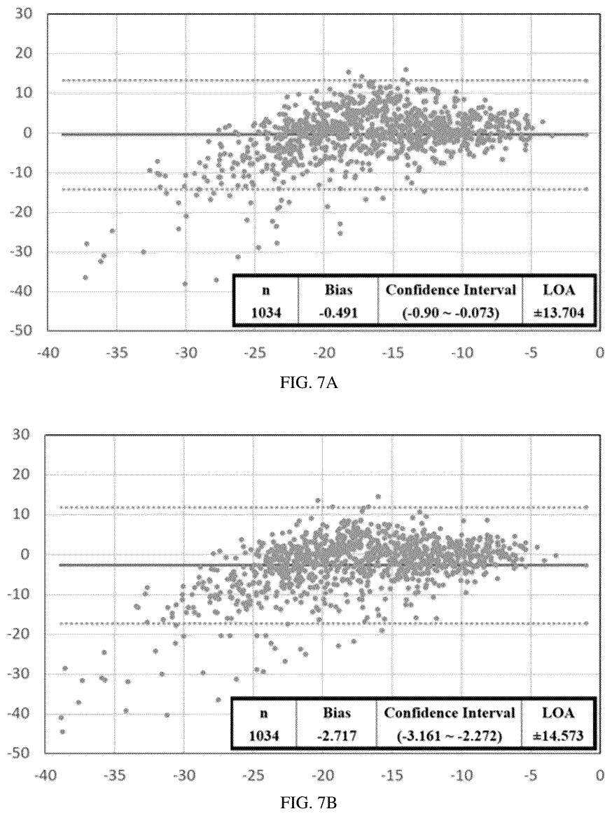

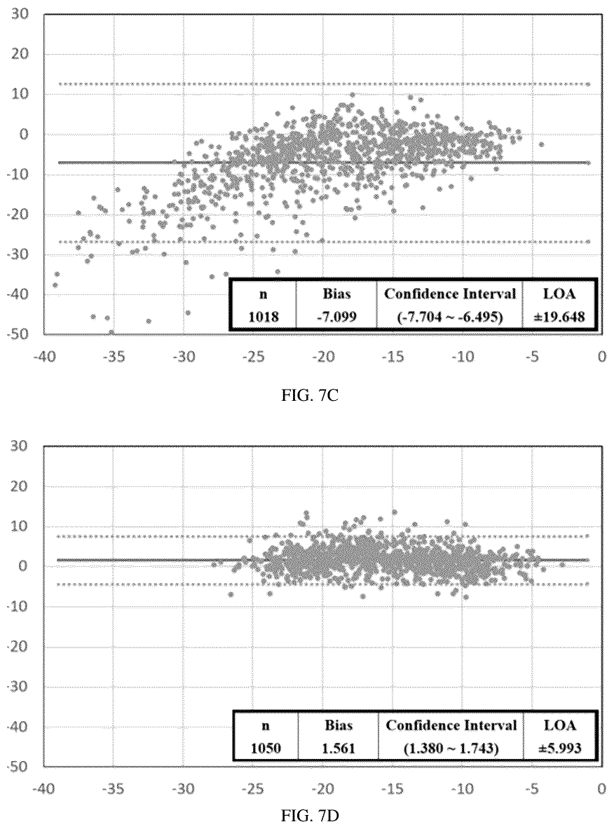

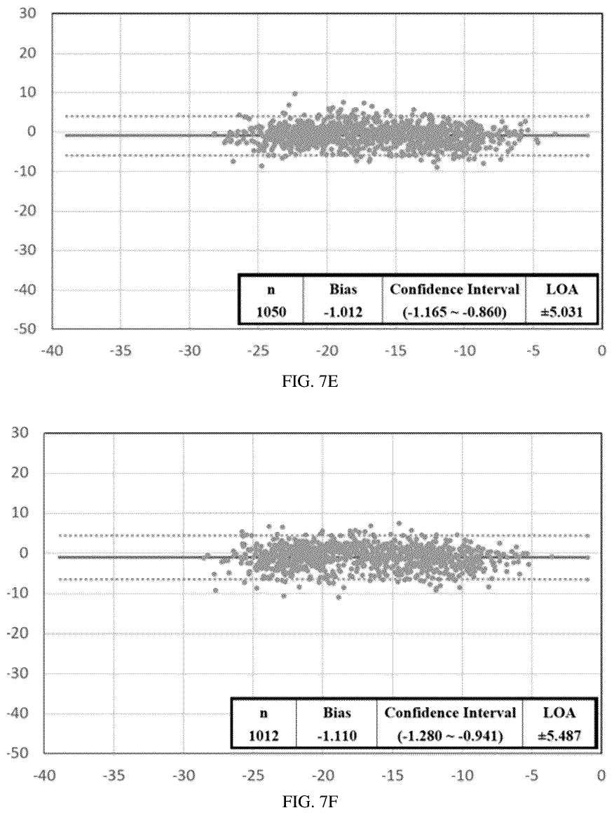

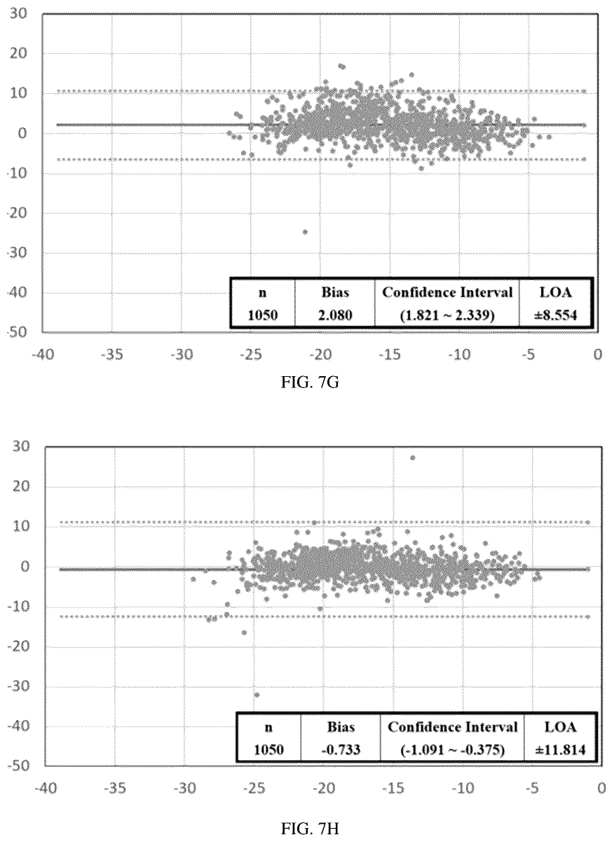

FIGS. 7A-7I summarizes the results of Bland-Altman analysis. All three proprietary combinations had small mean differences compared to expert strain, but DTHR+DTHR had the lowest limits of agreement (LOA) (bias, LOA: VSwinT-DP Strain −1.012, ±5.031; Unity-GLS −0.894, ±9.829; EchoNet-Dynamic: −0.491, ±13.704). The VSwinT contour maintains good agreement regardless of the strain calculation algorithm used (bias, LOA: VSwinT+EchoNet Strain 1.561, +5.993; VSwinT+Unity Strain −1.110, +5.487). However, the agreement of EchoNet-Dynamic and Unity-GLS decreases when using other strain algorithms (bias, LOA: EchoNet+DP Strain −2.717, +14.573; EchoNet+Unity Strain −7.099, +19.648; Unity+DP Strain −0.733, 11.814; Unity+EchoNet Strain 2.080, +8.554).

### ROC Analysis to Predict LVGLS Value Above/Below Normal Range

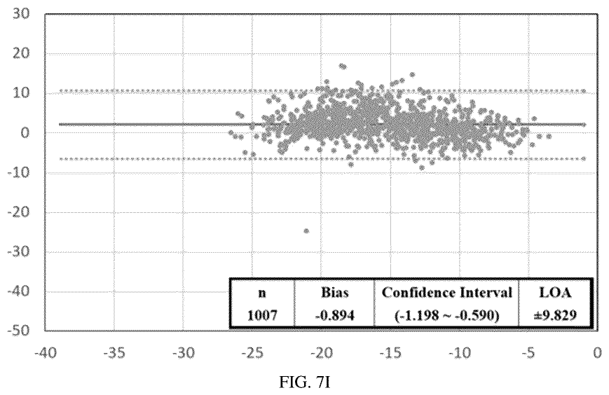

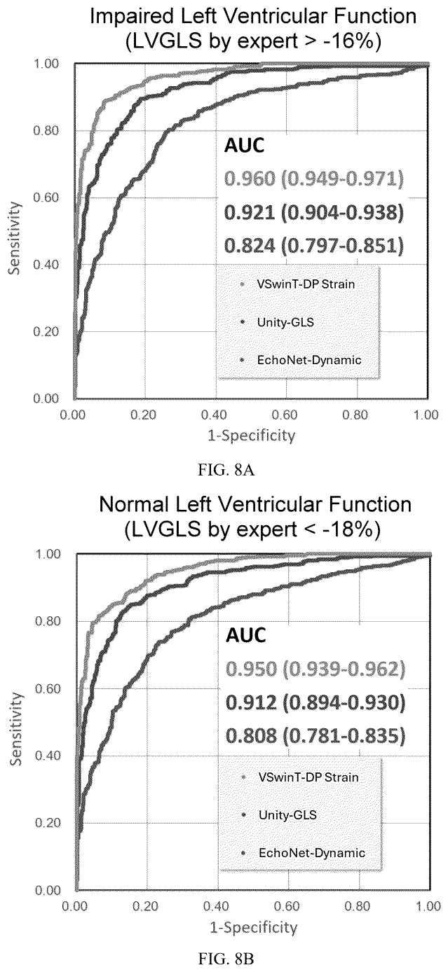

Clinically, a LVGLS value of less than-18% is considered normal, while a value greater than −16% is indicative of impaired LV function. ROC analysis was performed to evaluate the diagnostic performance of these three proprietary combinations of AI-based strain methods. To predict impaired LV function, the AUC values were 0.960 (0.949-0.971) for VSwinT-DP Strain, 0.921 (0.904-0.938) for Unity-GLS, and 0.824 (0.797-0.851) for EchoNet-Dynamic (FIG. 8A). To predict normal LV function, the AUC values were 0.950 (0.939-0.962) for VSwinT-DP Strain, 0.912 (0.894-0.930) for Unity-GLS, and 0.808 (0.781-0.835) for EchoNet-Dynamic (FIG. 8B).

### Application of VSwinT-DP Strain to A2C, A3C, and Averaged LVGLS

We evaluated the agreement between VSwinT-DP Strain derived LVGLS and expert reference values across apical 2-chamber (A2C), apical 3-chamber (A3C), and the averaged 3-view LVGLS. In the A2C view, VSwinT-DP Strain achieved Spearman's p of 0.8450 and an ICC of 0.833 (0.814-0.851), with a bias of 0.488 and LOA of =5.61. For the A3C view, the model yielded a p of 0.8585 and an ICC of 0.853 (0.836-0.896), with a slightly higher bias of 1.143 and LOA of =5.11. When averaging GLS across A4C, A2C, and A3C views, the model achieved the strongest correlation with expert values (p=0.9193, ICC=0.913 (0.903-0.923)), and the narrowest LOA (+3.51%), with a minimal bias of 0.21%.

### REAL EXAMPLES

The EchoNet-Dynamic model generates segmentation masks as outputs, which require additional computer vision algorithms to extract the LV contour from the masks. Similarly, the Unity-GLS model produces heatmaps corresponding to the contour regions, which are then processed using computer vision techniques to derive the LV contour.

In contrast, our proposed model directly predicts the precise 2D coordinates of the LV contour points, eliminating the need for intermediate conversion steps, thereby ensuring greater efficiency and accuracy.

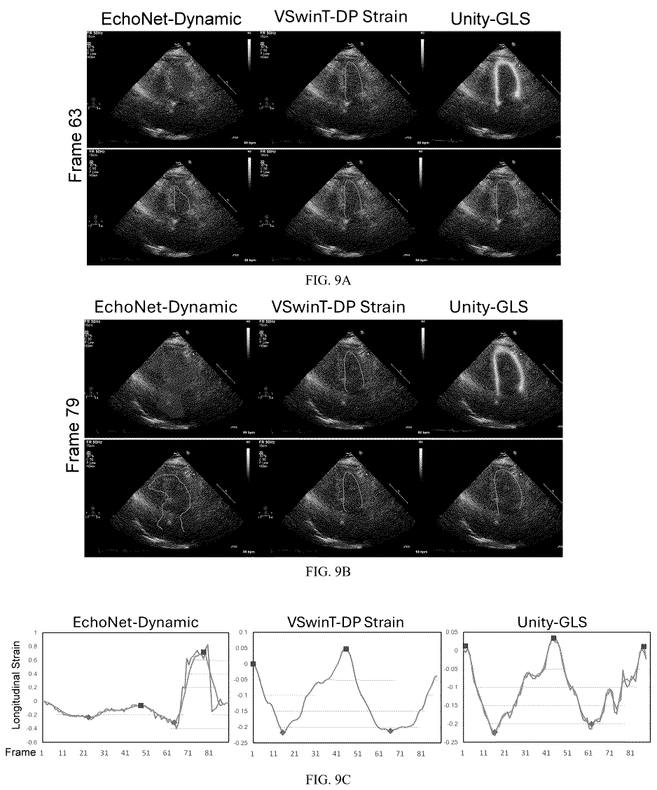

FIGS. 9A-9C shows performance of three AI models under poor image quality. Poor image quality results in irregular and temporally inconsistent LV segmentation masks in EchoNet-Dynamic (left panels), leading to highly jittery strain-to-time curves and errors in identifying ED and ES time points (FIG. 9C, left panel). Although Unity-GLS success fully computes LVGLS, the strain-to-time curves exhibit significant jittering (FIG. 9C, right panel, blue curve) that cannot be resolved even with smoothing filters (FIG. 9C, right panel, orange curve).

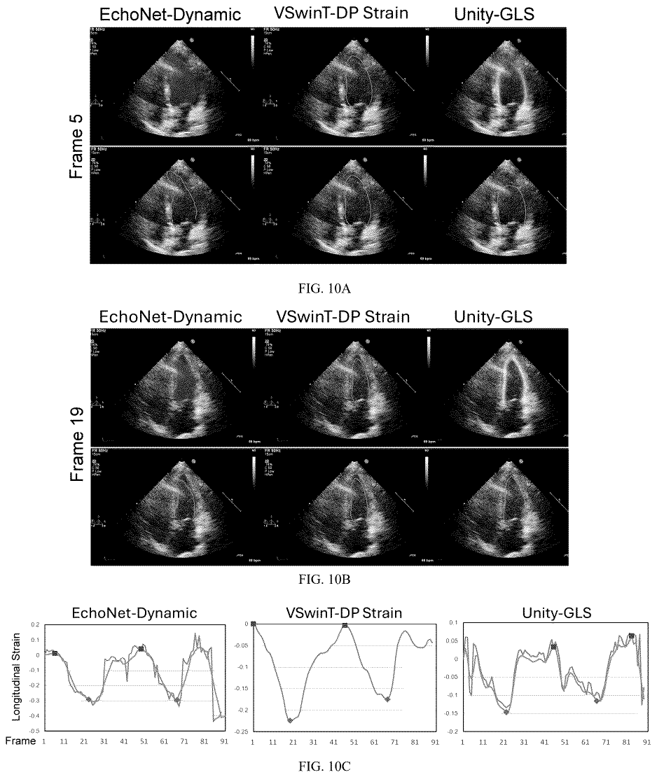

FIG. 10A-10C shows performance of three AI models under reverberation artifacts. Reverberation artifacts cause distorted LV mask in EchoNet-Dynamic (FIGS. 10A and 10B, upper-left panels, red mask) and truncated apex in Unity-GLS (FIG. 10A, upper-right panel, faded-out red shadow and yellow stripe at LV apex). These errors further affect the accuracy of myocardial length estimation, leading to erroneous strain-to-time curves (FIG. 10C, blue curves in left and right panels) that cannot be corrected by smoothing filters (FIG. 10C, orange curves in left and right panels).

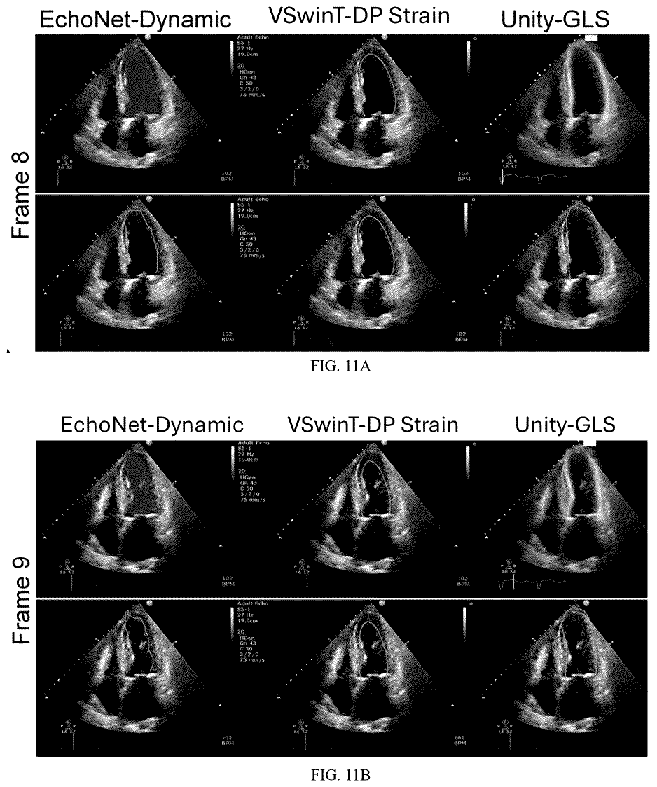

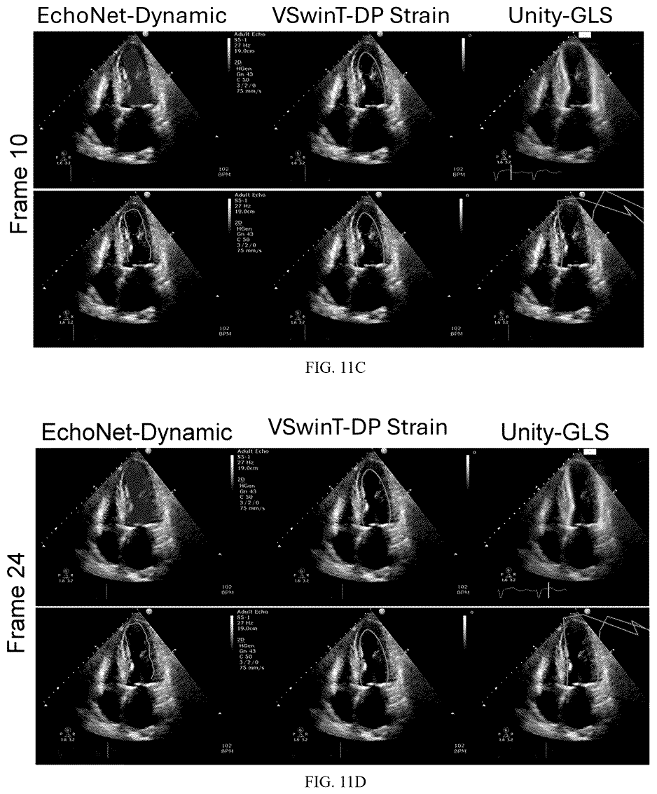

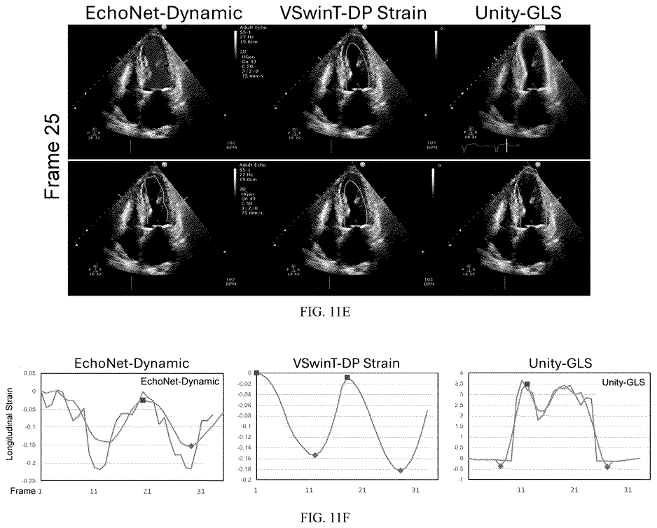

FIGS. 11A-11F shows performance of three AI models under poor delineation of LV apex. FIGS. 11A-11E are selected frames to demonstrate the segmentation performance of AI models. The Unity-GLS struggles with the low confidence of its LV border heatmap, resulting in noisy contour lines during LV border calculations (FIGS. 11C and 11D, right panels, yellow stripes and green contours). The temporal consistency of EchoNet-Dynamic deteriorates further in poor apical delineation (FIGS. 11A-11E, left panels, red masks and corresponding green contours), leading to a reduced number of ED/ES points identified by the strain algorithm (FIG. 11F). Both EchoNet-Dynamic and Unity-GLS failed identification of the end diastolic and end-systolic point of the strain-to-time curve (FIG. 11F, wherein errors exist in the number and placement of red (end-diastolic) and green (end-systolic) points on the left and right strain-curve panels). In this image, the blood-muscle border at the apical lateral wall is more easily identifiable in the video than in the static frame. And the spatiotemporal consistency of VSwinT-DP Strain effectively addresses this issue (FIGS. 11A-11F, middle panels).

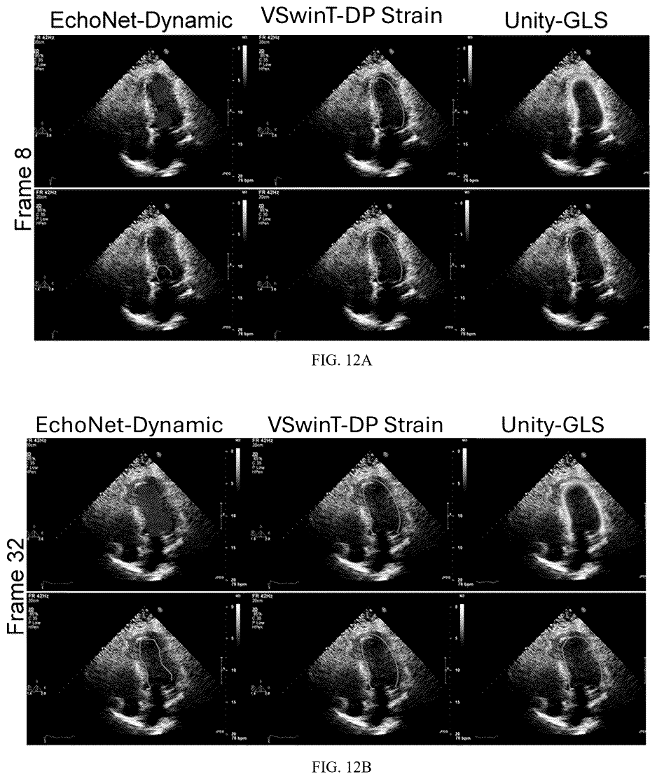

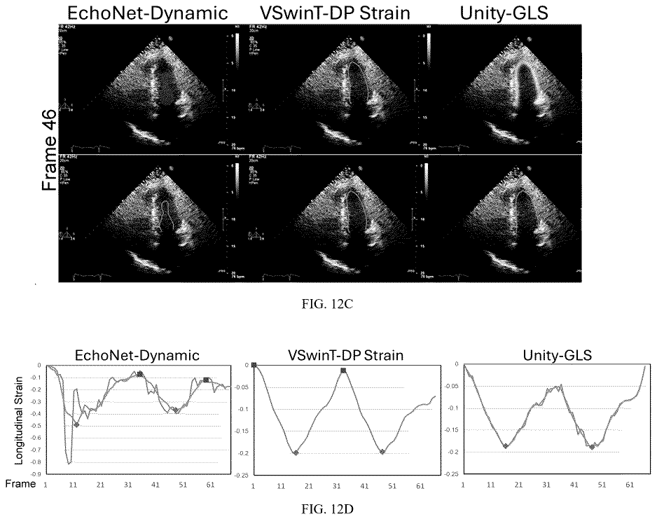

FIGS. 12A-12D shows an example with irrational end-diastolic or end-systolic time points due to a lack of temporal consistency. Due to the end-diastolic/end systolic threshold limitations in the Unity-GLS strain algorithm, no end-diastolic point was identified for this video (FIG. 12D, missing red dots in right curves), making LVGLS calculation impossible. Additionally, fragmented masks were observed during segmentation with EchoNet-Dynamic, resulting in a fragmented, irregular LV mask (FIGS. 12A-12C, upper-left panels) and an anatomically inconsistent contour (FIGS. 12A and 12C, bottom-left panels).

### Advantages of VSwinT and Regression-Based Design

Our framework uses a VSwinT backbone combined with a regression head and an FCN-style multiscale feature fusion. This design improves both computational efficiency and accuracy, addressing the limitations seen in prior methods such as EchoNet-Dynamic and Unity-GLS.

EchoNet-dynamic relies on a DeepLab V3+architecture that includes a CNN backbone, neck, and segmentation head. While this approach excels in extracting comprehensive segmentation maps for cardiac contours, it is computationally intensive and not optimized for direct strain calculation. Similarly, Unity-GLS uses HRNet architecture with a heatmap head for feature point localization. Although robust in many settings, heatmap predictions can occasionally fail, leading to positional errors (FIGS. 10A, 11C and 11D).

In contrast, our use of a regression head avoids post processing steps, eliminates heatmap quantization errors, and removes the need for manually designed ground truth heatmaps. Previous CNN-based regression approaches struggled with stability and precision, but VSwinT overcomes these issues. Our VSwinT-DP Strain not only reduces computational overhead but also effectively captures the cardiac shape and spatiotemporal relationships. These improvements restore the feasibility of regression heads in LVGLS calculation.

In addition, the FCN-style multiscale fusion in our architecture balances global context with fine local details. Unlike EchoNet-Dynamic's segmentation head, our regression head generates compact, parameterized outputs with minimal distortion. This makes our approach more efficient and more applicable in clinical settings.

### Importance of Spatiotemporal Consistency on Echocardiography

CNN-based neural networks have achieved significant success in static image semantic segmentation. This success naturally led to the use of 3D spatiotemporal convolutions for dynamic cardiac ultrasound. While this approach performs well in estimating LVEF in high-quality, preselected echocardiographic images, it faces limitations when applied to LVGLS computation.

Unlike traditional object segmentation, cardiac chambers are not distinct objects with clear boundaries. Instead, they are separated by thin, fast-moving valves or structures like the atrial-ventricular septum, which has inconsistent echogenicity. When each frame is segmented independently, the resulting pixel masks tend to have irregular, jagged borders and poor temporal coherence (FIGS. 9-12, red masks in left panels). This lack of temporal consistency causes instability in the strain-to-time curve. Researchers often apply smoothing filters (orange curves in FIGS. 9C, 10C, 11F and 12D) and rely on precise criteria for identifying ED and ES timepoints to reduce such fluctuations.

The performance of CNN-based segmentation is further limited by several operator-related factors, including foreshortened views, suboptimal acquisition angles, patient-related factors, including translational motion and occasional out-of-plane motion, and ultrasound-specific artifacts such as reverberation, refraction, side lobes, mirror artifacts, and near-field clutter. These challenges contribute to increased segmentation failure rates, as shown in FIGS. 9-12. For example, EchoNet-Dynamic often produces uneven contours (FIG. 9) and fragmented segmentation masks (FIG. 12), sometimes even including masks for other cardiac chambers or regions outside the heart. Unity-GLS also fails when the confidence of its LV border heatmap is low (FIG. 10), resulting in noisy contours (FIG. 11). These inaccuracies lead to excessive contour lengths that significantly distort strain values. To compensate, Unity-GLS applies length-based thresholds at ED and ES in its strain algorithm. However, this approach can sometimes abandon strain calculations (FIG. 12). This explains why Unity-GLS did not achieve a 100% contour-to-strain conversion rate in our experiments.

Transformer-based architectures leverage self-attention to effectively capture long-range dependencies, making them particularly suitable for echocardiographic image segmentation where cross-regional relationships and boundary ambiguities are common. Compared with convolutional neural networks, they provide stronger global contextual modeling, enhanced robustness to artifacts, and better integration of local and global features. Prior transformer-based approaches such as SegFormer and SwinT have demonstrated benefits in echocardiographic segmentation, but they are originally designed for static 2D frames, thus overlooking temporal dynamics. VSwinT addresses this limitation by extending SwinT's shifted window mechanism into three dimensions, incorporating the temporal axis. In this design, global self-attention enforces spatial consistency within each frame, while 3D shifted windows introduce temporal consistency across consecutive frames. Together, these mechanisms enable VSwinT to achieve robust spatiotemporal consistency, making it particularly well suited for echocardiographic video segmentation tasks. The present disclosure shows that VSwinT addresses these challenges with its superior spatiotemporal consistency. Consequently, the strain-to-time curves require no additional smoothing filters, simplifying strain algorithm design and reducing measurement variability.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded the widest scope consistent with the principles and novel features disclosed herein. It is contemplated that additional embodiments are within the spirit and true scope of the disclosed subject matter. Thus, it is intended that the present invention covers modifications and variations that come within the scope of the appended claims and their equivalents.

What is claimed is:

1. A computer implemented method for video-based image segmentation, comprising:
obtaining an input video comprising multiple image frames; and
generating predicted contour points of one or more target contours on one or more of the multiple image frames by a trained machine learning model;
wherein the trained machine learning model is trained with a training dataset prepared from multiple training videos, wherein each of the multiple training videos comprises multiple contiguous frames with annotated contour points of the one or more target contours.

2. The computer implemented method of claim 1, wherein the machine learning model comprises a feature extractor which applies local-window self-attention mechanism to both spatial and temporal dimensions to enhance segmentation stability across image frames.

3. The computer implemented method of claim 2, wherein the feature extractor is constructed based on a Video Swin Transformer (VSwinT) backbone.

4. The computer implemented method of claim 2, wherein the machine learning model further comprises a multi-scale feature fusion mechanism to generate a fused feature map from outputs of different stages of the feature extractor.

5. The computer implemented method of claim 4, wherein the machine learning model further comprises a contour regressor to generate the predicted contour points of the one or more target contours from the fused feature map.

6. The computer implemented method of claim 1, wherein the annotated contour points of the one or more target contours are used as training ground truth during training.

7. The computer implemented method of claim 1, wherein the machine learning model does not require mask-based segmentation or post-processing to generate the predicted contour points.

8. The computer implemented method of claim 1, wherein the input video is an echocardiogram video.

9. The computer implemented method of claim 8, wherein the one or more target contours comprise left ventricle (LV) endocardial line.

10. The computer implemented method of claim 9, wherein the one or more target contours further comprise left ventricle (LV) epicardial line and left atrium (LA) endocardial line.

11. The computer implemented method of claim 8, wherein the input video is an apical two-chamber view (A2C) echocardiogram video, an apical three-chamber view (A3C) echocardiogram video, or an apical four-chamber view (A4C) echocardiogram video.

12. The computer implemented method of claim 9, wherein the trained machine learning model generates predicted contour points on a plurality of contiguous image frames of the input video, and wherein the method further comprises:
for each of the plurality of the multiple image frames, calculating an left ventricular endocardium length from the predicted contour points of left ventricle (LV) endocardial line; and
identifying end-diastolic (ED) frames and end-systolic (ES) frames from the plurality of the multiple image frames based on the left ventricular endocardium length.

13. The method of claim 12, further comprising:
obtaining one or more ED-ES pairs by pairing each of the identified ED frames with a following ES frame selected from the identified ES frames;
calculating a longitudinal stain value for each of the one or more ED-ES pairs; and
averaging the longitudinal stain values of the one or more ED-ES pairs to obtain a final left ventricular longitudinal strain (LVLS).

14. The method of claim 12, wherein the end-diastolic (ED) frames and the end-systolic (ES) frames are identified by steps of:
obtaining an endocardium length series comprising the left ventricular endocardium lengths of the plurality of the multiple image frames;
obtaining an endocardium change series comprising differences of the left ventricular endocardium length between two consecutive image frames for each of the plurality of the multiple image frames;
based on the endocardium change series, determining a total state series which maximizes a penalized total path as an optimized state series; and
determining positions of the end-diastolic (ED) frames and the end-systolic (ES) frames based on the optimized state series; wherein:
the total state series comprises a state value describing systolic state or diastolic state at each time frame; and
the penalized total path is calculated based on the endocardium change series and the total state series, wherein a penalty value is introduced to the penalized total path every time when the state value changes in the total state series.

15. The method of claim 14, wherein the end-diastolic (ED) frames are the image frames where the state changes from diastolic state to systolic state, and wherein the end-systolic (ES) frames are the image frames where the state changes from systolic state to diastolic state in the optimized state series.

16. The method of claim 14, wherein the optimized state series is determined by a dynamic programming algorithm.

17. The method of claim 14, wherein said determining the optimized state series comprises iteratively calculating one or more penalized temporal paths and identifying one or more sub-optimal temporal paths from the one or more penalized temporal paths at each of the image frames.

18. A method to train a machine learning model concatenating a feature extractor, a multi-scale feature fusion mechanism and a contour regressor for generating contour points for video-based images, comprising:
obtaining a training dataset comprising multiple training data derived from multiple training videos, wherein each of the multiple training data comprises one of the multiple training videos as training input, and wherein each of the multiple training videos comprises a plurality of training image frames which are labeled with annotated contour points of one or more target contours as corresponding training ground truth;
inputting the training dataset into the machine learning model and performing parameter fitting to train the machine learning model, so that the machine learning model can use an input video comprising multiple image frames to generate predicted contour points of the one or more target contours for a plurality of the multiple image frames; wherein:
the feature extractor applies local-window self-attention mechanism to both spatial and temporal dimensions of the input video;
the multi-scale feature fusion mechanism receives outputs from different stages of the feature extractor generate a fused feature map based on the outputs; and
the contour regressor receives the fused feature map and generates the predicted contour points of the one or more target contours for the plurality of the multiple image frames based on the fused feature map.

19. The method of claim 18, wherein the feature extractor is constructed based on a Video Swin Transformer (VSwinT) backbone.

20. The method of claim 19, wherein the feature extractor generates three temporally intact feature tensors for the multi-scale feature fusion mechanism to generate the fused feature map.

21. The method of claim 18, wherein a mean squared error (MSE) loss function is used for parameter fitting of the machine learning model.

22. The method of claim 18, wherein each of the multiple training videos is an echocardiogram video.

23. The method of claim 22, wherein the one or more target contours comprise left ventricle (LV) endocardial line.

24. The method of claim 23, wherein the one or more target contours further comprise left ventricle (LV) epicardial line and left atrium (LA) endocardial line.
